(19) United States
(12) Patent Application Publication
(10) Pub. No.: US 2005/0101030 A1
(43) Pub. Date: May 12, 2005

(54) Methods of diagnosing the presence of trail

(76) Inventors: Pankaj J. Pasricha, Houston, TX (US); Nikolay K. Popnikolov, Philadelphia, PA (US); Massoud Motamedi, Houston, TX (US); Zoran Gatalica, Omaha, NE (US)

Correspondence Address: Karla M. Weyand;Rogalskyj & Weyand, PO Box 44, Livonia, NY, 14487-0044, US

(21) Appl. No.: 10/389,169

(22) Filed: Mar. 14, 2003

Related U.S. Application Data
(60) Provisional application No. 60/364,060, filed on Mar. 14, 2002.

(51) Int. Cl. G01N 33/543

(52) U.S. Cl. 436/518

(57) ABSTRACT

The present invention relates to methods for detecting the presence of TRAIL in cells. The invention also provides methods for identifying dysplastic or cancer cells, methods of identifying substances for use in treating dysplastic or cancer cells, as well as methods for making compounds that are useful in treating dysplastic or cancer cells.

This application claims priority of U.S. Provisional Application Ser. No. 60/364,060, filed Mar. 14, 2002, which is hereby incorporated by reference.

### FIELD OF THE INVENTION

The subject invention is directed generally to methods relating to diagnosing the presence of TRAIL in cells and to methods for diagnosing dysplasia and cancer cells.

### DETAILED DESCRIPTION OF THE DRAWING

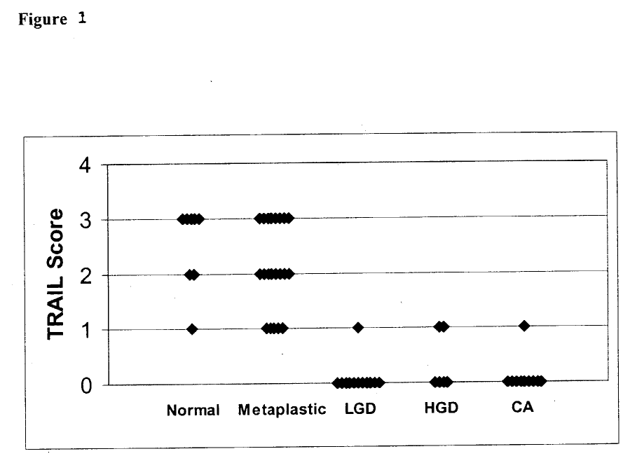

FIG. 1. TRAIL scores in normal gastro-esophageal (GE) junction (identified as Normal), metaplastic Barrett's mucosa without dysplasia (identified as Metaplastic), low-grade dysplasia (LGD), high-grade dysplasia (HGD), and adenocarcinoma (CA). TRAIL was always detected in the metaplastic Barrett's mucosa without dysplasia (100%) and the overall expression was similar to that of the normal GE junction. TRAIL was rarely and weakly (1+) expressed in Barrett's esophagus with dysplasia (16.7%) and adenocarcinoma (10.0%) (p<0.001).

### DETAILED DESCRIPTION OF THE INVENTION

Throughout this application various publications are referenced, many in parenthesis. The disclosures of each of these publications in their entireties are hereby incorporated by reference in this application.

One embodiment of the present invention relates to a method of detecting the presence of TRAIL in cells. The method includes contacting the cells with a compound which binds to TRAIL and determining whether TRAIL is present in the cells.

TNR-related apoptosis-inducing ligand (TRAIL), also known as Apo2L, is a type II transmembrane protein that was identified and cloned based on sequence homology with members of the tumor necrosis factor (TNF) ligand family. TRAIL is a CD95 ligand having 281 amino acids. TRAIL is a related member of the TNF family that initiates apoptosis in immune and neoplastic cells after binding to specific surface receptors.

In the methods of the present invention, compounds which bind to TRAIL are any compounds, such as peptides, antibodies, receptors or the like, which bind to TRAIL. As used herein, an antibody, peptide, receptor or the like, is said to “specifically bind” or “bind” to TRAIL if it reacts at a detectable level (within, for example, an ELISA) with TRAIL, and does not react detectably with unrelated proteins under similar conditions. As used herein, “binding” refers to a noncovalent association between two separate molecules such that a complex is formed. The ability to bind may be evaluated by, for example, determining a binding constant for the formation of the complex.

In one embodiment, the compound of the present invention includes an antibody. In one embodiment, the antibody is a monoclonal antibody, such as B35-1.

In alternative embodiments, monoclonal antibodies can be prepared which specifically bind to TRAIL.

The monoclonal antibodies can be produced by hybridomas. A hybridoma is an immortalized cell line which is capable of secreting a specific monoclonal antibody.

In general, techniques for preparing polyclonal and monoclonal antibodies as well as hybridomas capable of producing the desired antibody are well known in the art (see Campbell, A. M., “Monoclonal Antibody Technology: Laboratory Techniques in Biochemistry and Molecular Biology”, Elsevier Science Publishers, Amsterdam, The Netherlands (1984); St. Groth, et al., J Immunol Methods 35:1-21 (1980)). Any animal (mouse, rabbit, etc.) which is known to produce antibodies can be immunized with TRAIL (or an antigenic fragment thereof). Methods for immunization are well known in the art. Such methods include subcutaneous or intraperitoneal injection of TRAIL. One skilled in the art will recognize that the amount of TRAIL used for immunization will vary based on the animal which is immunized, the antigenicity of TRAIL, and the site of injection.

The TRAIL which is used as an immunogen may be modified or administered in an adjuvant in order to increase it's antigenicity. Methods of increasing the antigenicity of a protein are well known in the art and include, but are not limited to, coupling the antigen with a heterologous protein (such as a globulin or beta-galactosidase) or through the inclusion of an adjuvant during immunization.

For monoclonal antibodies, spleen cells from the immunized animals are removed, fused with myeloma cells, such as SP2/O-Ag 15 myeloma cells, and allowed to become monoclonal antibody producing hybridoma cells.

Any one of a number of methods well known in the art can be used to identify the hybridoma cell which produces an antibody with the desired characteristics. These include screening the hybridomas with an ELISA assay, western blot analysis, or radioimmunoassay (Lutz, et al., Exp Cell Res 175:109-124 (1988)).

Hybridomas secreting the desired antibodies are cloned and the class and subclass are determined using procedures known in the art (Campbell, A. M., “Monoclonal Antibody Technology: Laboratory Techniques in Biochemistry and Molecular Biology”, Elsevier Science Publishers, Amsterdam, The Netherlands (1984)).

For polyclonal antibodies, antibody containing antisera is isolated from the immunized animal and is screened for the presence of antibodies with the desired specificity using one of the above-described procedures.

Once a monoclonal antibody which specifically binds to, or hybridizes to, TRAIL is identified, the monoclonal antibody (which is itself a compound which can be used in the subject invention) can be used to identify peptides capable of mimicking the binding activity of the monoclonal antibody. One such method utilizes the development of epitope libraries and biopanning of bacteriophage libraries. Briefly, attempts to define the binding sites for various monoclonal antibodies have led to the development of epitope libraries. Parmley and Smith developed a bacteriophage expression vector that could display foreign epitopes on its surface (Parmley, S. F. & Smith, G. P., Gene 73:305-318 (1988)). This vector could be used to construct large collections of bacteriophage which could include virtually all possible sequences of a short (e.g. six-amino-acid) peptide. They also developed biopanning, which is a method for affinity-purifying phage displaying foreign epitopes using a specific antibody (see Parmley, S. F. & Smith, G. P., Gene 73:305-318 (1988); Cwirla, S. E., et al., Proc Natl Acad Sci USA 87:6378-6382 (1990); Scott, J. K. & Smith, G. P., Science 249:386-390 (1990); Christian, R. B., et al., J Mol Biol 227:711-718 (1992); Smith, G. P. & Scott, J. K., Methods in Enzymology 217:228-257 (1993)).

After the development of epitope libraries, Smith et al. then suggested that it should be possible to use the bacteriophage expression vector and biopanning technique of Parmley and Smith to identify epitopes from all possible sequences of a given length. This led to the idea of identifying peptide ligands for antibodies by biopanning epitope libraries, which could then be used in vaccine design, epitope mapping, the identification of genes, and many other applications (Parmley, S. F. & Smith, G. P., Gene 73:305-318 (1988); Scott, J. K., Trends in Biochem Sci 17:241-245 (1992)).

Using epitope libraries and biopanning, researchers searching for epitope sequences found instead peptide sequences which mimicked the epitope, i.e., sequences which did not identify a continuous linear native sequence or necessarily occur at all within a natural protein sequence. These mimicking peptides are called mimotopes. In this manner, mimotopes of various binding sites/proteins have been found.

The sequences of these mimotopes, by definition, do not identify a continuous linear native sequence or necessarily occur in any way in a naturally-occurring molecule, i.e. a naturally occurring protein. The sequences of the mimotopes merely form a peptide which functionally mimics a binding site on a naturally-occurring protein.

Many of these mimotopes are short peptides. The availability of short peptides which can be readily synthesized in large amounts and which can mimic naturally-occurring sequences (i.e. binding sites) offers great potential application.

Using this technique, mimotopes to a monoclonal antibody that recognizes TRAIL can be identified. The sequences of these mimotopes represent short peptides which can then be used in various ways, for example as peptides that bind to TRAIL. Once the sequence of the mimotope is determined, the peptide can be chemically synthesized.

The peptides for use in the subject invention can contain any naturally-occurring or non-naturally-occuring amino acids, including the D-form of the amino acids, amino acid derivatives and amino acid mimics, so long as the desired function and activity of the peptide is maintained. The choice of including an (L)- or a (D)-amino acid in the peptide depends, in part, on the desired characteristics of the peptide. For example, the incorporation of one or more (D)-amino acids can confer increased stability on a peptide and can allow a peptide to remain active in the body for an extended period of time. The incorporation of one or more (D)-amino acids can also increase or decrease the pharmacological activity of a peptide.

The peptide may also be cyclized, since cyclization may provide the peptide with superior properties over their linear counterparts.

Modifications to the peptide backbone and peptide bonds thereof are encompassed within the scope of amino acid mimic or mimetic. Such modifications can be made to the amino acid, derivative thereof, non-amino acid moiety or the peptide either before or after the amino acid, derivative thereof or non-amino acid moiety is incorporated into the peptide. What is critical is that such modifications mimic the peptide backbone and bonds which make up the same and have substantially the same spatial arrangement and distance as is typical for traditional peptide bonds and backbones. An example of one such modification is the reduction of the carbonyl(s) of the amide peptide backbone to an amine. A number of reagents are available and well known for the reduction of amides to amines such as those disclosed in Wann et al., JOC 46:257 (1981) and Raucher et al., Tetrahedron Lett 21:14061 (1980). An amino acid mimic is, therefore, an organic molecule that retains the similar amino acid pharmacophore groups as are present in the corresponding amino acid and which exhibits substantially the same spatial arrangement between functional groups.

The substitution of amino acids by non-naturally occurring amino acids and amino acid mimics as described above can enhance the overall activity or properties of an individual peptide thereof based on the modifications to the backbone or side chain functionalities. For example, these types of alterations can enhance the peptide's stability to enzymaticbreakdown and increase biological activity. Modifications to the peptide backbone similarly can add stability and enhance activity.

One skilled in the art, using the identified sequences can easily synthesize the peptides for use in the invention. Standard procedures for preparing synthetic peptides are well known in the art. The novel peptides can be synthesized using: the solid phase peptide synthesis (SPPS) method of Merrifield, J Am Chem Soc 85:2149 (1964) or modifications of SPPS; or, the peptides can be synthesized using standard solution methods well known in the art (see, for example, Bodanzsky, “Principles of Peptide Synthesis”, 2d Ed., Springer-Verlag (1993)). Alternatively, simultaneous multiple peptide synthesis (SMPS) techniques well known in the art can be used. Peptides prepared by the method of Merrifield can be synthesized using an automated peptide synthesizer such as the Applied Biosystems 431A-01 Peptide Synthesizer (Mountain View, Calif.) or using the manual peptide synthesis technique described by Houghten, Proc Natl Acad Sci USA 82:5131 (1985).

In the method of the present invention, TRAIL is detected in cells from any mammal. In one embodiment, TRAIL is detected in cells from humans. The cells may be endothelial cells, and in one embodiment the cells are from the gastrointestinal tract of the human. The gastrointestinal tract includes the esophagus, stomach, gastro-esophageal junction, small intestine and colon. In one embodiment, the cells are esophageal cells. Similarities in the topographic pattern of TRAIL expression in the normal gastro-esophageal junction, stomach, small intestine, and colon suggest a common function of TRAIL throughout the gastrointestinal tract. TRAIL expression is present in cells of the normal gastrointestinal tract (i.e. without dysplasia), such as gastro-esophageal cells without dysplasia. In addition, TRAIL is detected in Barrett's esophagus without dysplasia. Barrett's esophagus is a condition in which the esophagus changes so that some of its lining is replaced by a type of tissue similar to that normally found in the intestine. However, TRAIL expression is lost in dysplastic cells and/or cancer cells of the gastrointestinal tract. For example, TRAIL expression is lost in dysplastic cells and/or cancer cells of the esophagus, colon and gastro-esophageal junction. In another example, TRAIL expression is lost in the dysplastic cells and/or cancer cells of subjects having Barrett's esophagus.

Accordingly, in one embodiment of the present invention, the presence of TRAIL in cells of, for example the gastrointestinal tract, such as the esophagus, can be detected by contacting the cells with a compound which includes an antibody (or peptide or receptor, for example) which binds to the TRAIL present in the cells. The level of antibody (or peptide or receptor, for example) bound to the cells can be measured to determine the level of TRAIL expression in the cells. Thus, the absence of TRAIL in dysplastic or neoplastic cells or tissue can be used as a basis for a strategy to distinguish the dysplastic or neoplastic cells or tissue from normal cells or segments of tissue in a given specimen or subject.

The cells or tissue of the specimen or subject can be evaluated in vitro (via resected specimen, for example) or in vivo (via an endoscope to view fluorescence of the cells, for example).

In one embodiment, the antibody is conjugated to a diagnostic. The level of diagnostic is measured to determine the level of TRAIL expression in the cells.

Examples of diagnostics include, but are not limited to, markers such as fluorescent markers, radio-labeled, radioactive, calorimetric, or luminescent markers. Radioactive labels include, but are not limited to: ³H, ¹⁴C, ³²P, ³³P, ¹²⁵I, ¹³¹I, and ¹⁸⁶Re. Fluorescent markers include but are not limited to fluorescein, rhodamine and auramine. Colorimetric markers include, but are not limited to biotin and digoxigenin.

In another embodiment, the present invention relates to a method of identifying dysplastic or cancer cells. The method includes contacting the dysplastic or cancer cells with a compound which binds to TRAIL and identifying the dysplastic or cancer cells with substantially no compound bound thereto.

As used herein, “substantially no compound bound thereto” is defined as meaning no and/or trace amounts of compound bound to the cells of the present invention. As defined in the Examples below, dysplastic and/or cancer cells have no or weak expression of TRAIL, accordingly, no or minor amounts of compound which binds to TRAIL binds to the dysplastic or cancer cells.

Another embodiment of the invention relates to a method of identifying a substance which is useful in treating dysplastic or cancer cells of a subject. The method includes contacting the cells of the subject with a compound which binds to TRAIL, wherein the compound comprises the substance and determining whether the substance treats the cancer cells.

Another embodiment of the invention relates to a method for making a substance which is useful in treating dysplastic or cancer cells. The method includes carrying out the method of identifying a substance which is useful in treating dysplastic or cancer cells and manufacturing the substance.

Another embodiment of the invention provides a method of treating cells for dysplasia or cancer in a subject. In one embodiment, a method of the present invention is performed to identify cells which have substantially no TRAIL expression (i.e. cells which have substantially no compound bound thereto). The method includes administering to the subject an amount of a compound effective to reduce levels of cells having dysplasia or cancer in the subject. This can be accomplished by exposing the cells to a compound which includes a therapeutic, such as a radioactive or a toxin. Since the method of the subject invention is a method of treating cells for dysplasia or cancer, the subject can be an animal, such as a mammal, and can be a human.

The compounds used in the methods of the subject invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds and/or inhibitors used in the subject invention, pharmaceutically acceptable salts of such prodrugs, and other bioequivalents.

In regard to prodrugs, the compounds for use in the invention may additionally or alternatively be prepared to be delivered in a prodrug form. The term prodrug indicates a therapeutic agent that is prepared in an inactive form that is converted to an active form (i.e., drug) within the body or cells thereof by the action of endogenous enzymes or other chemicals and/or conditions.

In regard to pharmaceutically acceptable salts, the term pharmaceutically acceptable salts refers to physiologically and pharmaceutically acceptable salts of the compounds used in the subject invention: i.e., salts that retain the desired biological activity of the parent compound and do not impart undesired toxicological effects thereto.

In the context of this invention, to “contact” or “expose” cells (including the cells of tissues) to a compound, such as a compound including an antibody and a diagnostic, means to add the compound, in a liquid carrier, for example, to a cell suspension or tissue sample, either in vitro or ex vivo, or to administer the compound to cells or tissues within an animal (including a human) subject.

For therapeutics, methods of treating dysplastic and/or cancer cells are provided. The formulation of therapeutic compositions and their subsequent administration is believed to be within the skill in the art. In general, for therapeutics, a patient suspected of needing such therapy is given a compound in accordance with the invention, commonly in a pharmaceutically acceptable carrier, in amounts and for periods which will vary depending upon the nature of the particular disease, its severity and the patient's overall condition. The pharmaceutical compositions may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip or infusion, subcutaneous, intraperitoneal or intramuscular injection, pulmonary administration, e.g., by inhalation or insufflation, or intrathecal or intraventricular administration.

Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets or tablets. Thickeners, flavoring agents, diluents, emulsifiers, dispersing aids or binders may be desirable.

Compositions for parenteral, intrathecal or intraventricular administration may include sterile aqueous solutions which may also contain buffers, diluents and other suitable additives.

In addition to such pharmaceutical carriers, cationic lipids may be included in the formulation to facilitate oligonucleotide uptake. One such composition shown to facilitate uptake is Lipofectin (BRL, Bethesda Md.).

Dosing is dependent on severity and responsiveness of the condition to be treated, with course of treatment lasting from several days to several months or until a cure is effected or a diminution of disease state is achieved. Optimal dosing schedules can be calculated from measurements of drug accumulation in the body. Persons of ordinary skill can easily determine optimum dosages, dosing methodologies and repetition rates. Optimum dosages may vary depending on the relative potency of individual compounds and/or inhibitors, and can generally be calculated based on IC₅₀'s or EC₅₀'s in in vitro and in vivo animal studies. For example, given the molecular weight of compound (derived from oligonucleotide sequence and/or chemical structure) and an effective dose such as an IC₅₀, for example (derived experimentally), a dose in mg/kg is routinely calculated.

Given the known amino acid sequence of TRAIL, one skilled in the art can design appropriate compounds for use in the subject invention. Furthermore, by expressing the TRAIL in a host cell, one can screen for suitable compounds and/or inhibitors for use in the subject invention (see below for a further discussion of screening methods). The activity of TRAIL can be assayed according to methods known in the art.

Drugs, such as peptide drugs, which are useful in treating dysplastic and/or cancer cells can be made using various methods known in the art.

Materials and Methods

Methods: Immunohistochemical evaluation of TRAIL expression was performed on formalin-fixed paraffin sections from 29 gastroesophageal junction/esophageal biopsies, 20 gastric biopsies, 6 esophagectomy, 2 small bowel resection specimens, and 5 colon biopsies using B35-1 monoclonal antibody (Pharmimgen, San Diego, Calif.). The expression was graded semiquantitatively on a 4 point scale (0-3).

Immunohistochemistry: The immunoperoxidase stain was performed using an automatic immunostainer (DAKO Corp., Carpenteria, Calif.) and a streptavidin-biotin-peroxidase complex technique (LSAB2 system, DAKO). Heat-induced epitope retrieval was performed in a Handy Steamer Plus (Black and Decker) in citrate buffer, pH 6.0 (DAKO) for 20 minutes. The primary antibody against TRAIL (B35-1, Pharmingen, San Diego, Calif.) was employed at 1:400 dilution.

Scoring: The staining for TRAIL was graded on a 4 point scale (0-3) incorporating the intensity of staining and the percentage positive cells: 0, no staining; 1+, a weak speckled staining that does not entirely cover the appropriate cellular compartment, or an uniform staining of the appropriate compartment seen in less than 10% of cells; 2+, a complete but moderate staining of the appropriate compartment in more that 10% of cells; 3+, a complete and strong staining in the appropriate cellular compartment seen in more than 10% of cells.

Statistical analysis: Statistical comparisons were made with the χ²-test. A p value of less than 0.05 was considered to be significant.

### EXAMPLE

The purpose of the example was to study TRAIL/Apo2L expression in normal gastroesophageal (GE) junction, Barrett's esophagus with and without dysplasia, and associated adenocarcinoma.

The pattern of TRAIL expression during the malignant transformation of Barrett's esophagus is unknown. A specific topographic pattern of TRAIL expression in normal colonic mucosa and the loss of TRAIL expression in tubular adenomas as well as the majority of carcinomas of the colon has been reported [14]. Here TRAIL expression in normal gastroesophageal (GE) junction, Barrett's esophagus with and without dysplasia, and associated adenocarcinoma was studied.

TRAIL was expressed in the foveolar epithelium of normal GE junction and stomach as well as in the normal intestinal epithelium with maximal expression in the surface epithelium. TRAIL was always detected in Barrett's esophagus (100%) and the overall expression was similar to that of the normal gastroesophageal junction. TRAIL was rarely and weakly (1+) expressed in Barrett's esophagus with dysplasia (16.7%) and adenocarcinoma (10.0%) (p<0.001).

Similarities in the topographic pattern of TRAIL expression in the normal gastroesophageal junction, stomach, small intestine, and colon suggest a common function of TRAIL throughout the gastrointestinal tract. Down regulation of TRAIL is associated with development of dysplasia in Barrett's esophagus. Thus, alterations of the TRAIL apoptotic pathway appear to play a significant role in the progression from Barrett's metaplasia to carcinoma and may have diagnostic, prognostic, and potential therapeutic significance.

During the past decades the incidence and mortality rates of esophageal adenocarcinoma have been rising steadily in the western world [1]. Although esophageal adenocarcinoma has been associated with gastroesophageal reflux disease and the development of metaplastic specialized intestinal epithelium (Barrett's esophagus), the molecular events responsible for the metaplasia-carcinoma sequence are poorly understood. In general, defects in either proliferation or apoptosis have been implicated in tumor initiation, progression, and metastasis [2] and several molecules have been identified with the potential of playing an important role in these pathways. One of these is the TNF-related apoptosis-inducing ligand (TRAIL) or Apo2L, which is capable of activating the extrinsic apoptotic pathway by binding to specific receptors [3, 4]. TRAIL mRNA is detected in a variety of normal tissues including small intestine, colon, prostate, ovary, peripheral blood lymphocytes, spleen, and thymus [4]. It has been shown that TRAIL induces apoptosis in different tumorogenic or transformed cells but not in normal cells [4] and this is accomplished by activation of caspases pathways [5]. Two different types of TRAIL receptors have been discovered: receptors containing a cytoplasmic ‘death domain’ (TRAIL-R1 and TRAIL-R2) [6-9] capable of transmitting apoptosis signal and decoy receptors (TRAIL-R3 and TRAIL-R4) [7, 8, 10-12] which do not transmit a death signal and can prevent the induction of apoptosis via TRAIL-R1 and TRAIL-R2. Unlike the death receptors (TRAIL-R1 and TRAIL-R2) that are found in both normal and tumor cells, the decoy receptors are expressed mostly in normal tissues and by few tumor cells. However, it seems likely that multiple intra- and extracellular factors determine the effect of TRAIL on target cells [13].

Results

TRAIL Expression in the Normal Esophagus, Stomach and Intestine

TRAIL was not expressed in the squamous esophageal. TRAIL was detected in the foveolar epithelium of the stomach and normal GE junction with the immunoreactivity located mostly on the basolateral membranes. Some cytoplasmic immunoreactivity was also seen. TRAIL immunostaining was also noted in some of the gastric neuroendocrine cells. No difference in TRAIL immunoreactivity patterns was found in different topographic areas of the stomach. In the small intestine TRAIL was located in the surface epithelium of the distal two thirds of the villi. The crypt epithelium was negative. The intestinal absorptive cells showed strong membranous and cytoplasmic TRAIL expression. The intestinal Goblet's cells lacked obvious immunoreactivity. In the colon TRAIL expression was observed in the upper third of the crypts with maximum at the luminal surface. The pattern of staining was membranous and cytoplasmic with lack of immunoreactivity in the areas with accumulated intracellular mucin.

TRAIL Expression in Barrett's Esophagus

The pattern of TRAIL staining in metaplastic Barrett's mucosa was similar to that noted in the normal small intestinal mucosa. TRAIL expression in the columnar esophageal mucosa is summarized in FIG. 1. TRAIL was always detected in Barrett's esophagus without dysplasia (100%) and the overall expression was similar to that of the normal GE junction. TRAIL was expressed only in 16.7% of Barrett's esophagus with dysplasia and in those cases the overall expression was low (1+). Loss of TRAIL expression was found in both low grade and high grade dysplasia. Adenocarcinomas showed weak TRAIL positivity (1+) in 10.0% of the cases. Adenocarcinomas and Barrett's esophagus with dysplasia showed statistically significant decrease in TRAIL expression when compared with metaplastic mucosa without dysplasia (p<0.001).

The physiological function of TRAIL in the normal gastrointestinal tract is not completely understood. TRAIL is expressed on the luminal surface of the non-squamous compartment of the digestive tract. Multiple studies utilizing different techniques have shown that the bulk of apoptosis occurs mostly in this particular location [15-17]. TRAIL and death receptors (TRAIL-R1 and TRAIL-R2) are co-localized in the cells from the upper part of colonic crypts and colonic surface epithelium, while TRAIL-R4 is localized in the TRAIL-negative basal portions of the crypts [18]. However, although the normal colonic epithelial cells express death receptors, they are completely resistant to TRAIL-induced apoptosis in vitro [18]. This phenomenon can be explained by the presence of an intracellular inhibitory system. This system includes the cellular FADD-like IL-1β-converting enzyme (FLICE)-inhibitory protein, which can inhibit TRAIL or FAS-ligand induced apoptosis by binding of the death effector domain (DED) of Fas-associated death domain (FADD) protein.

Findings from several studies have outlined some possible aspects of TRAIL function. Strater et al [18] have shown that adenovirus infection increases TRAIL sensitivity of colon cancer cell and up-regulates TRAIL-R1 and TRAIL-R2 on cell surface. They have suggested that in the intestine the TRAIL system may participate in the elimination of virus infected enterocytes. Other studies have shown that the TRAIL pathway contributes to γ-radiation induced apoptosis. Radiation induces TRAIL and TRAIL-R2 expression in T-cell leukemia [19]. Genetically altered leukemia cells expressing nonfunctional TRAIL-R2 death domain have significantly augmented survival after treatment with y-radiation. Zhou et al [20] have shown that radiation induces also TRAIL-R2 expression and inhibits colonization of immortal non-tumorigenic human breast epithelial cells. Further antibody inhibition studies have confirmed that the inhibition of colonization is mediated via the TRAIL/Apo2L pathway.

An interesting relationship exists between interferons and TRAIL. Interferons induce TRAIL expression in many inflammatory cells. This has led to the suggestion that the antitumoral effect of interferons may be, at least partially, mediated via TRAIL-induced killing of tumor cells [21 and references herein]. Interferons γ and α also induce TRAIL expression and suppress cell growth in non-inflammatory cells including colon adenocarcinoma cell lines in vitro [22-27]. On the other hand, studies on breast cancer have shown that TRAIL induces multiple genes related to the interferon signaling pathway, including signal transducer and activator of transcription 1 (STAT1) [28], which is a known mediator of antiproliferative effect of interferons γ and α [29]. Taken together these findings suggest that TRAIL may produce some of the interferon effects, such as cell growth inhibition.

TRAIL expression is downregulated in the majority of dysplastic or neoplastic tissue associated with columnar lined esophagus. By contrast, Barrett's esophagus without dysplasia demonstrated a pattern of TRAIL expression similar to the normal gastroesophageal junction. TRAIL apoptotic system has an important role for anticancer defense and surveillance in Barrett's esophagus. TRAIL is induced at a certain stage of maturation of normal epithelial cells and may contribute to their growth suppression, making them less vulnerable to cytotoxic agents. In addition, some harmful agents, such as viruses, radiation or other carcinogens, may also activate the TRAIL apoptotic pathway by influencing death receptor expression or the TRAIL inhibitory system, leading to destruction of the affected cells via autocrine or paracrine mechanisms. On the other hand, down-regulation or absence of TRAIL may be an important factor in the development of genetic instability and accumulation of gene mutations, leading to dysplasia and eventually, carcinoma. This assumption is also supported by the fact that down-regulation of TRAIL expression even in low-grade dysplasia was observed, suggesting that this event occurs early in the transformation sequence.

The mechanism of TRAIL down regulation is obscure. No inactivating mutations in TRAIL gene has been reported in tumors, although cytogenetic abnormalities involving 3q26, which harbors the TRAIL gene, have been found in adenocarcinomas of esophagus, stomach and colon (The Cancer Genome Anatomy Project, http://cgap.nci.nih.gov). Rao et al [30] have reported that del (3q) is the most common cytogenetic abnormality in the gastric and esophageal carcinomas included in their study. Reviewing the literature, they concluded that 33% of those tumors show chromosomal changes affecting the 3q11-q27 region.

In summary, down regulation of TRAIL is associated with development of dysplasia in Barrett's esophagus. Thus, alterations of the TRAIL apoptotic pathway appear to play a significant role in the progression from Barrett's metaplasia to carcinoma.

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the claims which follow.

### REFERENCES

1. Vizcaino A P, Moreno V, Lambert R. et al. Time trends incidence of both major histologic types of esophageal carcinomas in selected countries, 1973-1995. Int J Cancer 2002; 99:860-868.
2. Evan G I and Vousden K H. Proliferation, cell cycle, and apoptosis in cancer. Nature 2001, 411: 342-348.
3. Pitti R M, Marsters S A, Ruppert S, et al. Induction of apoptosis by APO-2 ligand, a new member of the tumor necrosis factor cytokine family. J Biol Chem 1996; 271: 12687-12690.
4. Wiley S R, Schooley K, Smolak P J, et al. Identification and characterization of a new member of the TNF family that induces apoptosis. Immunity 1995; 3: 673-682.
5. Mariani S M, Matiba B, Armandola E A, et al. Interleukin 1 beta-converting enzyme related proteases/caspases are involved in TRAIL-induced apoptosis of myeloma and leukemia cells. J Cell Biol 1997; 137: 221-229.
6. Pan G, O'Rourke, Chinnaiyan A M, et al. The receptor for the cytotoxic ligand TRAIL. Science 1997; 276: 111-113.
7. Pan G, Ni J, Wei Y-F, et al. An antagonist decoy receptor and a death domain-containing receptor for TRAIL. Science 1997; 227: 815-818.
8. Sheridan J P, Marsters S A, Pitti P M, et al. Control of TRAIL-induced apoptosis by a family of signaling and decoy receptors. Science 1997; 277: 818-821.
9. Walczak H, Degli-Esposti, Johnson R S, et al. TRAIL-R2: a novel apoptosis-mediating receptor for TRAIL. EMBO J. 1997; 16: 5386-5397.
10. Degli-Esposti M A, Smolak P J, Walczac H., et al. Cloning and characterization of TRAIL-R3, a novel member of the emerging TRAIL receptor family. J Exp Med 1997; 186: 1165-1170.
11. Degli-Esposti M A, Dougall W C, Smolak P J, et al. The novel receptor TRAIL-R4 induces NF-kB and protects against TRAIL-mediated apoptosis, yet retains and incomplete death domain. Immunity 1997; 7: 813-820.
12. Marsters S A, Sheridan J P, Pitti P M, et al. A novel receptor for Apo2/TRAIL contains truncated death domain. Curr Biol 1997; 7: 1003-1006.
13. Griffith T S and Lynch D H. TRAIL: a molecule with multiple receptors and control mechanisms. Curr Opin Immunol 1998, 10: 559-563.
14. Popnikolov, N, Norris B A, Pasricha, et al. TRAIL/APO2L Expression during colonic carcinogenesis and its correlations with CD95/FAS and p53. Mod Pathol 2001; 14: 93A.
15. Crossman J, Walther K, Artinger M, et al. Induction of apoptosis before shedding of human intestinal epithelial cells. Am J Gastroenterol 2002; 97: 1421-1428.
16. Lehmann F S, Terracciano L, Carena I, et al. In situ correlation of cytokine secretion and apoptosis in Helicobacter pylory-associated gastritis. Am J Physiol Gastrointest Liver Physiol 2002; 283: G481-G488.
17. Moss S, Scholes J V, Holt P R. Abnormalities of epithelial apoptosis in multystep colorectal neoplasia demonstrated by terminal deoxyuridine nick end labeling. Dig Dis Sci 1996; 41: 2238-2247.
18. Strater J, Walczak H, Pukrop T, et al. TRAIL and its receptors in the colonic epithelium: a putative role in the defense of viral infections. Gastroenterology 2002; 122: 659-666.
19. Gong B and Almasam A. Apo2 ligand/TNF-related apoptosis-inducing ligand and death receptor 5 mediate the apoptotic signaling induced by ionizing radiation in leukemia cells. Cancer Res 2002; 60: 5754-6760.
20. Zhou Q, Fukushima P, DeGraff W, et al. Radiation and Apo2L/TRAIL apoptotic pathway preferentially inhibit the colonization of premalignant human breast epithelial cells overexpressing cyclin D1. Cancer Res 2000; 60: 2611-2615.
21. Walczak H and Krammer P H. The CD95 (APO-1/FAS) and TRAIL (APO-2L) apoptosis systems. Exp Cell Res, 2000; 256:58-66.
22. Gong B and Almasam A. Genomic organization and transcriptional regulation of human Apo2/TRAIL gene. Biochem Biophys Res Commun 2000; 278: 747-752.
23. Inoue H, Shiraki K, Yamanaka T, et al. Functional expression of tumor necrosis factor-related apoptosis-inducing ligand in human colonic adenocarcinoma cells. Lab Invest 2002; 82: 1111-1119.
24. Orlovski K, Teodor L, Malovani K, et al. Gamma interferon (IFN-gamma) down-regulates Fer and induces its association with inactive STAT3 in colon carcinoma cells. Oncogene 2002; 21: 4997-5001.
25. Pfizenmaier K, Bartch H, Scheurich P et al. Differential gamma-interferon response of human colon carcinoma cells: inhibition of proliferation and modulation of immunogenicity as independent effect of gamma-interferon on tumor cell growth. Cancer Res 1985; 45: 3503-3509.
26. Susuki S, Mita S, Kamohara H, et al. IL-6 and INF-gamma regulation of IL-10 production by human colon carcinoma cells. Int J Oncol 2001; 18: 581-586.
27. Wang Q, Ji Y, Wang X, et al. Isolation and molecular characterization of the 5′-upsteam region of the human TRAIL gene. Biochem Biophys Res Commun 2000; 276: 466-471.
28. Kumar-Sinha C, Varambally S, Sreekumar A, et al. Molecular cross-talk between the TRAIL and interferon signaling pathways. J Biol Chem 2002; 277: 575-585.
29. Bromberg J F, Horvath C M, Wen Z, et al. Transcriptiomally active Stat1 is required for the antiproliferative effects of both interferon γ and interferon α. Proc Natl Acad Sci USA 1996; 93: 7673-7678.
30. Rao P H, Mathwe S, Kelsen D P, et al. Cytogenetics of gastric and esophageal adenocarcinomas. Cancer Genet Cytogenet 1995; 81: 139-143.

1. A method of detecting the presence of TRAIL in cells, the method comprising:
contacting the cells with a compound which binds to TRAIL and
determining whether TRAIL is present in the cells.

2. A method according to claim 1, wherein the compound comprises an antibody.

3. A method according to claim 1, wherein the compound comprises a diagnostic.

4. A method according to claim 3, wherein the diagnostic is a fluorescent marker.

5. A method according to claim 4 wherein the determining step comprises measuring the fluorescence of the compound bound to the cells.

6. A method according to claim 5 wherein the cells are endothelial cells.

7. A method according to claim 6 wherein the cells are esophageal cells.

8. A method according to claim 7 wherein the compound comprises antibody B35-1.

9. A method of identifying dysplastic or cancer cells, the method comprising:
contacting the dysplastic or cancer cells with a compound which binds to TRAIL and
identifying the dysplastic or cancer cells with substantially no compound bound thereto.

10. A method according to claim 9, wherein the compound comprises an antibody.

11. A method according to claim 9, wherein the compound comprises a diagnostic.

12. A method according to claim 11, wherein the diagnostic is a fluorescent marker.

13. A method according to claim 12 wherein the identifying step comprises measuring the fluorescence of the compound bound to the dysplastic or cancer cells.

14. A method according to claim 13 wherein the cells are endothelial cells.

15. A method according to claim 14 wherein the cells are esophageal cells.

16. A method according to claim 15 wherein the compound comprises antibody B35-1.

17. A method of identifying a substance which is useful in treating dysplastic or cancer cells, the method comprising:
contacting the cells of the subject with a compound which binds to TRAIL, wherein the compound comprises the substance and
determining whether the substance treats the cancer cells.

18. A method for making a substance which is useful in treating dysplastic or cancer cells, the method comprising:
carrying out the method of claim 17 to identify the substance; and
manufacturing the substance.

19. A method of treating a subject having dysplastic or cancer cells comprising:
performing the method of claim 1;
identifying the cells having substantially no TRAIL expression and
contacting the cells having substantially no TRAIL expression with a therapeutic.

20. A method of treating a subject having dysplastic or cancer cells comprising:
performing the method of claim 9 and
contacting the dysplastic or cancer with substantially no compound bound thereto with a therapeutic.
